(19) United States
(12) Patent Application Publication
(10) Pub. No.: US 2004/0171860 A1
(43) Pub. Date: Sep. 2, 2004

(54) Novel acylating reagents

(76) Inventors: Hong Zhao, Edison, NJ (US); Richard B. Greenwald, Somerset, NJ (US)

Correspondence Address: MUSERLIAN, LUCAS & MERCANTI, LLP, 15th Floor, 475 Park Avenue South, New York, NY, 10016, US

(21) Appl. No.: 10/705,741

(22) Filed: Nov. 11, 2003

Related U.S. Application Data
(60) Provisional application No. 60/425,893, filed on Nov. 12, 2002.

(51) Int. Cl. C07C 69/96; C07C 329/02

(52) U.S. Cl. 558/245; 558/275; 558/248

(57) ABSTRACT

Novel acylating agents useful for preparing activated nucleophiles such as terminally activated polymers are disclosed. In preferred aspects, the acylating agents are

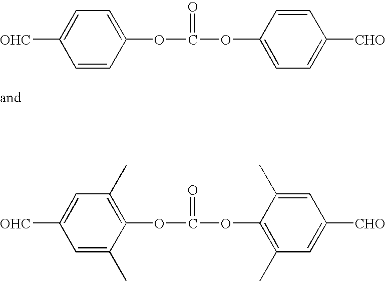

and they used to prepare activated polymers such as PEG's for conjugating small molecules, proteins, polypeptides and the like.

### CROSS REFERENCE TO RELATED APPLICATION

This application claims the benefit of priority from U.S. provisional patent application No. 60/425,893 filed Nov. 12, 2002, the contents of which are incorporated herein by reference.

### FIELD OF THE INVENTION

The present invention relates to novel acylating reagents. Specifically, the invention provides such reagents for preparing, inter alia activated polymer linkers for various biologically active compounds made with the activated polymers.

### BACKGROUND OF THE INVENTION

PEGylation has been shown as an important way to improve various properties of biologically active materials such as polypeptides, enzymes and small molecules such as camptothecin or paclitaxel. Typically, attachment of one or more strands of polyethylene glycol (hereinafter PEG) to a biologically active target has been shown to increase the solubility of the target, increase the circulating life of the target in vivo, and decrease the immunogenicity of the target. Initially, Davis et al. in U.S. Pat. No. 4,179,337 disclosed conjugating PEG to polypeptides, such as enzymes and insulin. In order to facilitate attachment of the polymer to the target, Davis et al. described attaching specific functional groups to a terminal of the linear PEG. Such reactions are generally referred to as activation reactions and they are the key to linking the PEG to the target. Early PEGylation reactions almost exclusively focused on linking the polymer to an epsilon amino group of a lysine found on the target polypeptide, etc.

Over the years, various improvements to PEG technology have been offered to expand its utility. One of the early forms of activated PEG used in the art was cyanuric chloride-activated PEG. While many demonstrated that this type of activated PEG could be used to make conjugates, it was found to suffer from drawbacks associated with its hapten (toxic) degradation products. Zalipsky, in commonly assigned U.S. Pat. No. 5,112,614, disclosed formation of succinimidyl carbonate activated PEG's using phosgene. The activated PEG, often referred to as SC-PEG, was designed to form carbamate (urethane) linkages with epsilon amino groups of proteins, enzymes and the like without forming toxic degradation products.

Greenwald et al. have disclosed several improvements in PEG technology. For example, commonly-assigned U.S. Pat. No. 5,349,001 discloses cyclic imide thione-activated PEG's which have improved hydrolytic stability. U.S. Pat. No. 6,113,906 discloses branched PEG derivatives which employ a wide variety of leaving groups as a means of increasing polymer loading while minimizing the points of attachment.

More recently, Greenwald et al. have disclosed multipart prodrug platforms for releasably attaching higher molecular weight PEG's (i.e. >20,000) to amino and hydroxyl-containing small molecules. After administration to a patient, the polymer portion hydrolyzes at a predetermined rate due to the inclusion of preselected bifunctional linkers. Once the polymer portion has been hydrolyzed, a cyclization reaction is initiated or triggered which thereafter rapidly releases the parent compound. See, U.S. Pat. No. 6,180,095 which discloses benzyl elimination (BE) systems and U.S. Pat. Nos. 5,965,119 and 6,303,569 which each disclose prodrug systems containing trimethyl lock triggers.

As an outgrowth of the foregoing, there has been interest in developing improvements in preparing the activated forms of PEG. U.S. Pat. No. 5,281,698 to Nitecki, for example, discloses the use of disuccinimidyl carbonate as an acylating agent for preparing activated succinimidyl carbonate activated PEG's. Although some artisans believe that this method has advantages over the phosgene-based systems, further improvements have been sought. Traditional acylating reagents continue to produce low yields and poor quality products. The present invention provides an alternative to the foregoing and therefore provides a solution to these shortcomings.

### SUMMARY OF THE INVENTION

In one aspect of the present invention, acylating agents corresponding to formula (I) are provided:

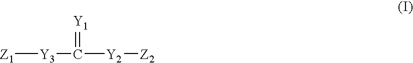

wherein:

Y₁₋₃ are independently O, S or NR₁;

Z₁, and Z₂ are independently selected substituted or unsubstituted aromatic hydrocarbons such as benzene or napthalene or substituted or unsubstituted heterocyclic aromatic groups such as pyridine, and which contain and aldehyde or protecting group; and

R₁ is selected from the group consisting of hydrogen, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxys, phenoxys and C₁₋₆ heteroalkoxys.

In another aspect of the invention, there are provided methods of preparing activated polymers or activated small molecule nucleophiles using the above described acylating agents. The methods include reacting a compound corresponding to formula (I) with a strong nucleophile, such as a polymer or a small molecule containing a primary or secondary amine under conditions sufficient to form a compound of the formula (II):

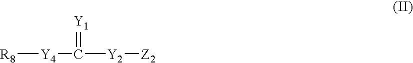

wherein:

R₈ is a residue of the strong nucleophile;

Y₄ is NR₂₀, O or S; wherein R₂₀ is selected from the same group as that which defines R₁.and all other variables are the same as that mentioned with regard to Formula (I).

Further aspects of the invention include converting the polymer-containing compounds of Formula (II) into activated polymers, i.e. polymers containing a leaving group which is capable of reacting with an amino group found on a polypeptide or therapeutic (small) molecule. Still further aspects of the invention include forming polymer conjugates containing a drug such as vancomycin or a polypeptide with the activated polymers formed with the methods and acylating agents described herein.

For purposes of the present invention, the term “residue” shall be understood to mean that portion of a biologically active compound which remains after it has undergone a substitution reaction.

For purposes of the present invention, the term “polymer containing residue” or “PEG residue” shall each be understood to mean that portion of the activated polymer or PEG which remains after it has undergone a substitution reaction with either a moiety containing leaving group or biologically active compound.

For purposes of the present invention, the term “alkyl” shall be understood to include straight, branched, substituted, e.g. halo-, alkoxy-, nitro-, C₁₋₁₂ alkyls, C₃₋₈ cycloalkyls or substituted cycloalkyls, etc.

For purposes of the present invention, the term “substituted” shall be understood to include adding or replacing one or more atoms contained within a functional group or compound with one or more different atoms.

For purposes of the present invention, substituted alkyls include carboxyalkyls, aminoalkyls, dialkylaminos, hydroxyalkyls and mercaptoalkyls; substituted alkenyls include carboxyalkenyls, aminoalkenyls, dialkenylaminos, hydroxyalkenyls and mercaptoalkenyls; substituted alkynyls include carboxyalkynyls, aminoalkynyls, dialkynylaminos, hydroxyalkynyls and mercaptoalkynyls; substituted cycloalkyls include moieties such as 4-chlorocyclohexyl; aryls include moieties such as napthyl; substituted aryls include moieties such as 3-bromo-phenyl; aralkyls include moieties such as toluyl; heteroalkyls include moieties such as ethylthiophene; substituted heteroalkyls include moieties such as 3-methoxy-thiophene; alkoxy includes moieties such as methoxy; and phenoxy includes moieties such as 3-nitrophenoxy. Halo- shall be understood to include fluoro, chloro, iodo and bromo.

The term “sufficient amounts” for purposes of the present invention shall mean an amount which achieves a therapeutic effect as such effect is understood by those of ordinary skill in the art.

The new acylating reagents will react with nucleophiles such as non-antigenic polymers containing at least one terminal amine under much milder conditions to give purer products in higher yield. This was an unmet need in the isolation of non-antigenic polymers.

### BRIEF DESCRIPTION OF THE DRAWINGS

FIGS. 1-5 describe various reactions set forth in the Examples which are employed to synthesize compounds in accordance with the present invention.

### DETAILED DESCRIPTION OF THE INVENTION

In one preferred embodiment of the invention, there are provided compounds of the formula:

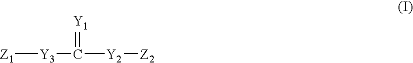

wherein:

Y₁₋₃ are independently O, S or NR₁;

Z₁, and Z₂ are independently selected substituted or unsubstituted aromatic hydrocarbons such as benzene or napthalene or substituted or unsubstituted heterocyclic aromatic groups such as pyridine containing an aldehyde or protecting group; and

R₁ is selected from the group consisting of hydrogen, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxys, phenoxys and C₁₋₆ heteroalkoxys.

Z₁, and Z₂ can be the same or different. In preferred aspects, Z₁, is

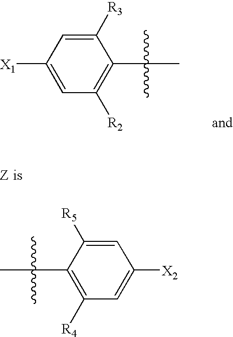

wherein:

X₁ and X₂ are independently selected from among:

CHO,

NO₂,

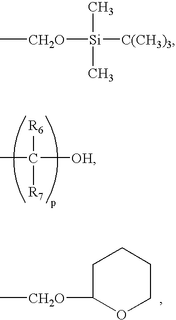

and other acetals generally corresponding to the formula:

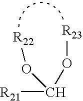

wherein R₂₁₋₂₃ are selected from the same group as that which defines R₁ and R₂₂ and R₂₃ optionally together form a heterocyclic group with the other members of the acetal group;

R₂₋₇ are independently selected from the group consisting of hydrogen, C₁₋₆ alkyls, C₃₋₈ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxys, phenoxys and C₁₋₆ heteroalkoxys; and

p is a positive integer, which is preferably 1.

With regard to other variables which comprise formula (I), the following are preferred: Y₁₋₃ are each O; R₂₋₇ are independently hydrogen or a C₁₋₆ alkyl; X₁ and X₂ are both CHO; and Z₂ is the same as Z₁. Furthermore, in many preferred aspects of the invention, X₁ and X₂ are the same. In other aspects, however, they are different which can be advantageous when one of Z₁, or Z₂ is more hindered.

There are several ways to prepare compounds corresponding to Formula (I). In one preferred method, a 4-hydroxy substituted or unsubstituted benzaldehyde is reacted with an acylating agent such as triphosgene, diphosgene, phosgene or an activated chloroformate such as paranitrophenylchloroformate, in the presence of a base such as diisopropylamine (DIEA), triethylamine (TEA), dimethylaminopyridine (DMAP), pyridine, etc.

In still another method, the compounds of formula (I) are prepared by first reducing a 4-hydroxy substituted or unsubstituted benzaldehyde with NaBH₄, NaCNBH₄ or other similar reducing agents in the presence of a protonic solvent such as methanol or ethanol. Alternatively, LiAlH₄ or other similar reducing agents can be used in non-protonic solvents such as tetrahydrofuran (THF). After the corresponding alcohol has been formed, it is reacted with a protecting group such as tetrahydropuran (THP), t-butyl-dimethylsilylchloride, other suitable silyl chlorides such as t-butyl-diphenylsilylchloride. This intermediate is then reacted with the acylating agent, triphosgene, etc. in the presence of a base.

A non-limiting list of suitable 4-hydroxy bezaldehydes include:

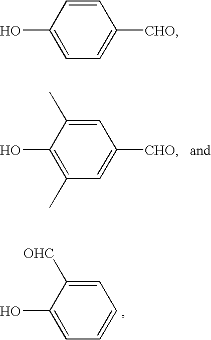

such compounds are available from Aldrich.

Typically, 4-hydroxy benzaldehyde is used as the bi-functional aromatic compound. Other aromatic compounds useful in the methods of the invention include but are not limited to 3-hydroxy benzaldehyde and 3 or 4-hydroxy benzyl alcohol. It will be understood by those or ordinary skill that the aromatic portions of such compounds may have multiple substitution and that protection or blocking of pivotal functional groups where necessary, will be accomplished without undue experimentation.

Preferably, the aromatic portions of the compounds useful in the methods of the invention are of the general formula:

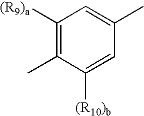

wherein R₉₋₁₀ are independently selected from the same group which defines R₁ and (a) and (b) are independently zero or one.

Other preferred aromatic hydrocarbon portions include, without limitation

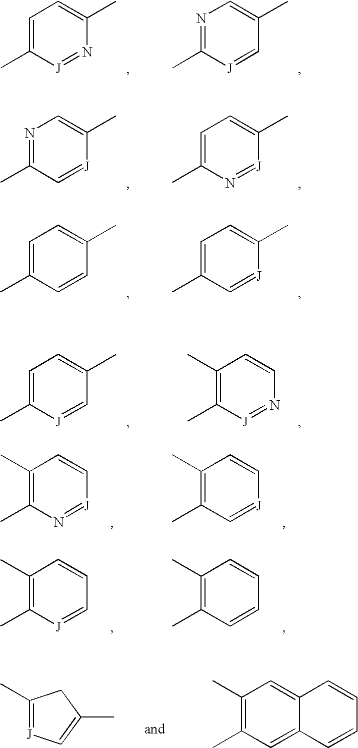

wherein J is CR₁₁ or NR₁₂; and

R₁₁₋₁₂ are independently selected from the same group as that which defines R₁ in formula (I) e.g., hydrogen, C₁₋₆ alkyls, etc. Isomers of the five and six-membered rings are also contemplated as well as benzo- and dibenzo-systems and their related congeners are also contemplated. It will also be appreciated by the artisan of ordinary skill that aromatic rings can optionally be substituted with hetero-atoms such as O, S, NR₁₂, etc. so long as Hockel's rule is obeyed. Furthermore, the aromatic or heterocyclic structures may optionally be substituted with halogen(s) and/or side chains as those terms are commonly understood in the art.

Preparation of preferred compounds corresponding to Formula (I) are set forth in the Examples. Some preferred compounds corresponding to this embodiment include:

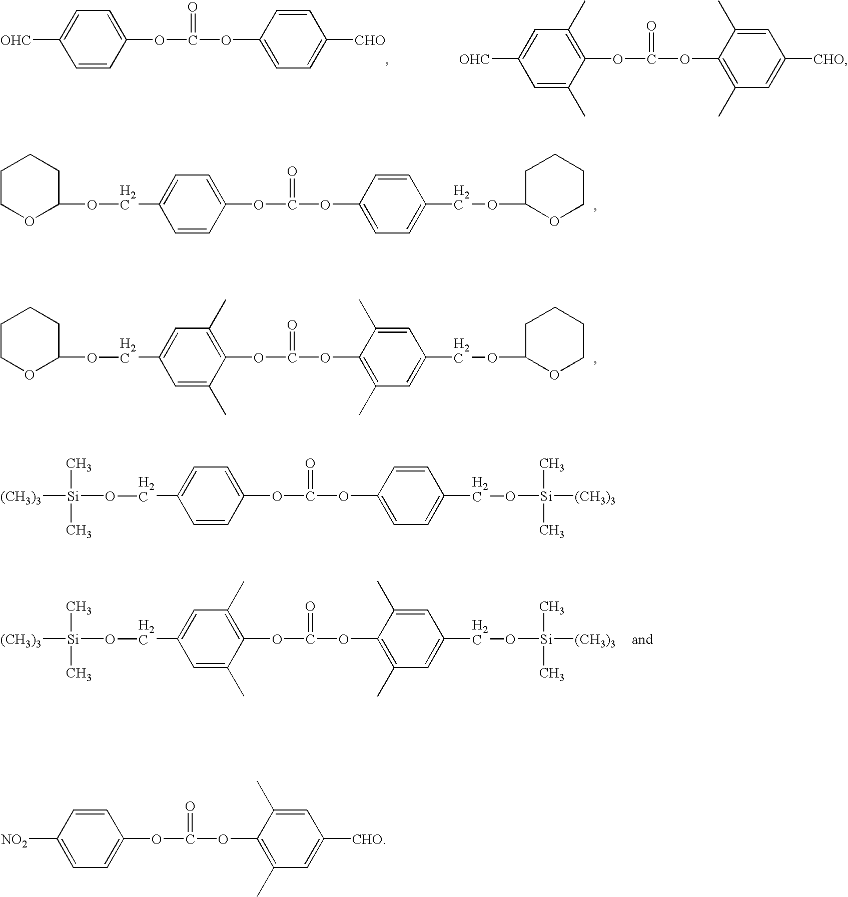

In another aspect, the invention provides methods of preparing activated polymers or activated small molecule nucleophiles using compounds of Formula (I). The methods include

a) reacting a compound of the formula:

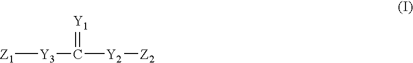

wherein all variables are as previously defined above, with a strong nucleophile, such as a polymer or a small molecule containing a primary or secondary amine, hydroxyl, thiol, etc. under conditions sufficient to form a compound of the formula (II):

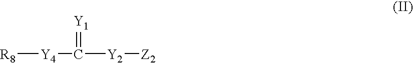

wherein:

R₈ is a residue of a strong nucleophile;

Y₄ is NR₂₀ O or S; wherein R₂₀ is selected from the same group as that which defines R₁;

Z₂ is a substituted or unsubstituted aromatic hydrocarbon or a substituted or unsubstituted heterocyclic aromatic group containing an aldehyde or protecting group; and

R₁ is selected from the group consisting of hydrogen, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxys, phenoxys and C₁₋₆ heteroalkoxys.

The conditions which are sufficient for carrying out the above reaction include carrying out the reaction in the presence of a solvent such as methylene chloride and a base such as DIEA at about room temperature.

Turning now to compounds of Formula (II), Z₂ is preferably

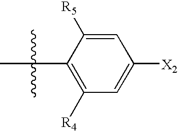

wherein:

X₂ is one of:

—CHO,

—NO₂

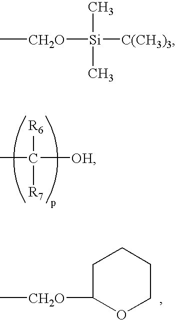

and other acetals as described above,

R₄₋₇ are independently selected from the group consisting of hydrogen, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxys, phenoxys and C₁₋₆ heteroalkoxys; and

p is a positive integer, such as from about 1 to about 10, more preferably 1.

In a still further aspect of the invention, when X₂ is not

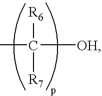

the compounds of Formula (II) are reduced or converted from their respective aldehyde or protecting group to provide their corresponding alcohol.

In yet a further aspect of the invention, with compounds of Formula (I) where Z₂ is preferably:

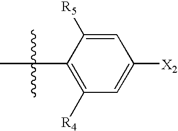

wherein:

X₂ is one of:

—CHO, NO₂

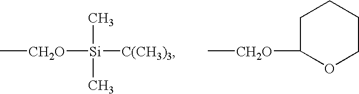

and other acetals as described above

X₂ is converted to an alcohol, thereby forming a compound of the formula:

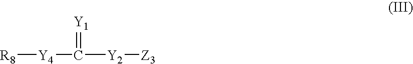

wherein Z₃ is a substituted aromatic hydrocarbon or a substituted heterocyclic aromatic group substituted with

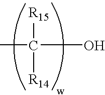

wherein R₁₅₋₁₆ are independently selected from the same group which defines R₆ and w is a positive integer, preferably 1 .

Standard reduction techniques, i.e. treatment with NaBH₄, etc. or deprotection techniques, i.e. using an acid such as acetic acid or hydrochloric acid are employed using standard techniques. Once the alcohol derivative has been formed, the polymer derivative of Formula (III) can be converted or activated using moieties containing leaving groups well known to those of ordinary skill to form compounds corresponding to Formula (IV):

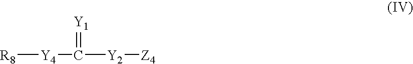

where Z₄ is a leaving group and all other variables are as previously defined.

A non-limiting list of suitable leaving groups which may be employed as Z₄ include groups such as without limitation, groups such as N-hydroxysuccinimidyl; N-hydroxybenzotriazolyl, halogen, N-hydroxyphthalimidyl, p-nitrophenoxy, imidazolyl, thiazolidinyl thione, O-acyl ureas, pentafluorophenol or 2,4,6-trichlorophenol. Other suitable leaving groups will be apparent to those of ordinary skill.

For purposes of the present invention, leaving groups are to be understood as those groups which are capable of reacting with an amino group (nucleophile) found on a target such as a small molecule containing an available amino group such as vancomycin, a polypetide, etc.

Such leaving groups are attached to the compounds of the present invention using standard coupling reactions. For example, a compound of Formula (III) can be reacted with disuccinimidyl carbonate (DSC) in the presence of pyridine. Other leaving groups can be added in similar fashion e.g. N-hydroxyl pthalamidyl groups can be obtained by reacting with di-N-hydroxylpthalamidyl carbonate in the presence of pyridine.

In some aspects of the invention, R₈ is a non-polymeric nucleophile containing a primary or secondary amine. For purposes of illustration and not limitiation, such nucleophiles include C₁₋₆ alkyl amines, benzyl amines and other, aromatic amines, etc. Such small molecules can be used as protecting groups in organic synthesis or to introduce certain special functional groups into a molecule such as a carbamate linkage.

In other preferred aspects, R₈ includes a polymer residue which is preferably water soluble at room temperature and is preferably substantially non-antigenic. A non-limiting list of such polymers include polyalkylene oxides, including homopolymers such as polyethylene glycol (PEG) (most preferred) or polypropylene glycols, polyoxyethylenated polyols, copolymers thereof and block copolymers thereof, provided that the water solubility of the block copolymers is maintained. In other preferred aspects, R₈ further includes a capping group on one terminal of the polymer, such as a C₁₋₆ alkyl, and more preferably a methyl.

As an example, the PEG residue portion of the inventive compositions can be selected from the non-limiting list:

A-O—(CH₂CH₂O)ₓ—

A-O—(CH₂CH₂O)ₓ—CH₂C(O)—O—,

A-O—(CH₂CH₂O)ₓ—CH₂CH₂NR₁₅—,

A-O—(CH₂CH₂O)ₓ—CH₂CH₂S,

—O—(CH₂CH₂O)ₓ—

—O—C(O)CH₂—O—(CH₂CH₂O)ₓ—CH₂C(O)—O—,

—NR₁₅CH₂CH₂—O—(CH₂CH₂O)ₓ—CH₂CH₂NR₁₅— and

—SCH₂CH₂—O—(CH₂CH₂O)ₓ—CH₂CH₂S—,

wherein

x is the degree of polymerization;

R₁₅ is selected from the group consisting of hydrogen, C₁₋₆ alkyls, C₃₋₁₂ branched alkyls, C₃₋₈ cycloalkyls, C₁₋₆ substituted alkyls, C₃₋₈ substituted cyloalkyls, aryls substituted aryls, aralkyls, C₁₋₆ heteroalkyls, substituted C₁₋₆ heteroalkyls, C₁₋₆alkoxy, phenoxy and C₁₋₆ heteroalkoxy and A is a capping group, preferably methyl.

For the purpose of the present invention the structure:

—O(CH₂CH₂O)ₓ

wherein x is a positive integer, is referred to as PEG throughout the application. The degree of polymerization for the polymer (x) can be from about 10 to about 2,300. This represents the number of repeating units in the polymer chain and is dependent on the molecular weight of the polymer.

Also useful for the polymer portion R₈ are branched PEG derivatives such as-those described in commonly-assigned U.S. Pat. No. 5,643,575, “star-PEG's” and multi-armed PEG's such as those described in Shearwater Corporation's 2001 catalog “Polyethylene Glycol and Derivatives for Biomedical Application”. The disclosure of each of the foregoing is incorporated herein by reference.

In certain further aspects, such as when R₈ includes a polymer, R₈ can be functionalized for attachment to a bifunctional extender or spacer group if desired without undue experimentation using standard coupling techniques. In such aspects, a compound of the Formula (IIa) is formed:

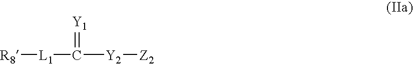

wherein:

R₈′ is a residue of R₈ which has undergone a substitution reaction with an activated bifunctional linker;

L₁ is a bifunctional spacer or linking moiety and all other variables are as described above.

For example, R₈ can be reacted with L₁-J wherein L₁, is preferably selected from among:

—C(O)CH₂OCH₂Y₅—;

—NHC(CH₃)₂CH₂Y₅—;

—C(O)CH₂CH₂Y₅—;

—C(O)CH₂Y₅—;

—NHCH₂(CH₃)Y₅—;

—NHCH₂Y₅—;

—NHCH₂CH₂Y₅—;

—NHCH₂CH₂OCH₂CH₂OY₅—;

—NHCH₂CH₂OCH₂CH₂OCH₂CH₂Y₅— and

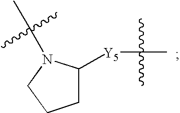

and

J is moiety capable of facilitating the reaction with a terminal group of R₈. For purposes of illustration, in one such reaction, a PEG-NH₂ is reacted with, β-alanine and the PEG-β-alanine is reacted with a compound of Formula (I) to yield a compound of formula (II).

Although PAO's and PEG's can vary substantially in average molecular weight, R₈ can have a weight average molecular weight of from about 20,000 Da to about 100,000 Da and more preferably from about 25,000 Da to about 60,000 Da. In general, the average molecular weight of the polymer selected for inclusion in the conjugate portion must be sufficient so as to provide sufficient circulation of a prodrug before hydrolysis of the linker.

In a further embodiment, and as an alternative to PAO-based polymers, R₈ is optionally selected from among one or more effectively non-antigenic materials such as dextran, polyvinyl alcohols, carbohydrate-based polymers, hydroxypropyl-methacrylamide (HPMA), polyalkylene oxides, and/or copolymers thereof. See also commonly-assigned U.S. Pat. No. 6,153,655, the contents of which are incorporated herein by reference. It will be understood by those of ordinary skill that the same type of activation is employed as described herein as for PAO's such as PEG. Those of ordinary skill in the art will further realize that the foregoing list is merely illustrative and that all polymeric materials having the qualities described herein are contemplated. For purposes of the present invention, “effectively non-antigenic” and “substantially non-antigenic” shall be understood to include all polymeric materials understood in the art as being substantially non-toxic and not eliciting an appreciable immune response in mammals.

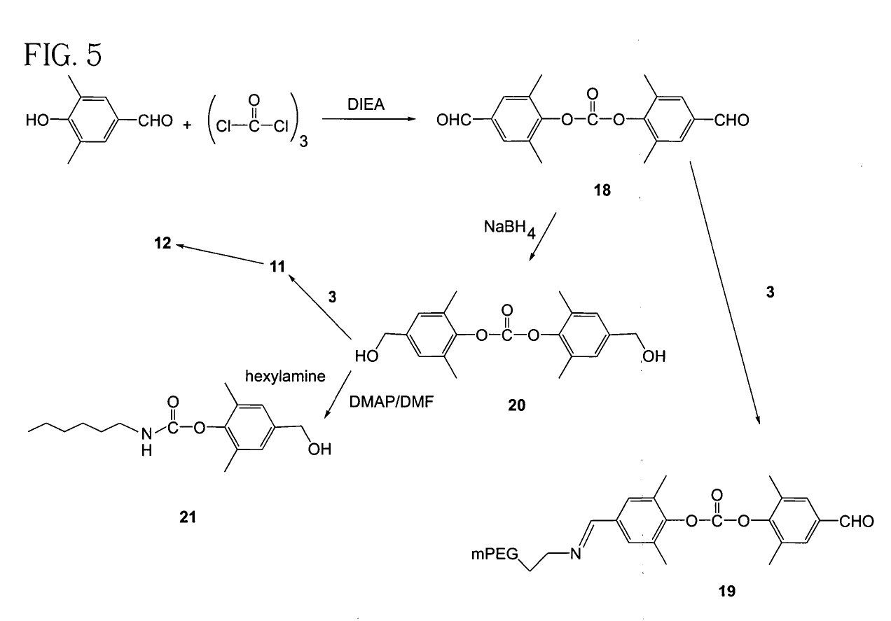

In a still further aspect of the invention, an alternative method of preparing an activated nucleophile, e.g. polymer, is provided. This method is also described in the Examples with regard to FIG. 5. First, a bis aldehyde corresponding to Formula (I) is reduced to form the corresponding alcohol derivative. In this aspect, such compounds correspond to the formula:

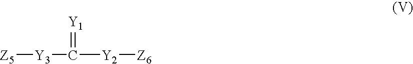

wherein:

Y₁₋₃ are independently O, S or NR₁;

Z₅ and Z₆ are independently selected substituted or unsubstituted aromatic hydrocarbons such as benzene or napthalene or substituted or unsubstituted heterocyclic aromatic groups such as pyridine, substituted with

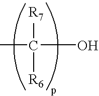

wherein

R₁ and R₆₋₇ are independently selected from the group consisting of hydrogen, C₁₋₆ alkyls, C₃₋₈ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxys, phenoxys and C₁₋₆ heteroalkoxys; and

p is a positive integer, preferably one.

The compound of Formula (V) is then reacted with a strong nucleophile such as a polymer containing primary or secondary amine or other nucleophile described above, under conditions sufficient to form a compound of the formula (II):

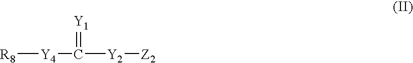

wherein:

R₈ is a residue of the strong nucleophile, and all other variables are the same as provided above.

Once the activated polymer has been formed, it can be reacted with any number of biologically active compounds, including those materials which have physiological or pharmacological activities as well as those which are able to catalyze reactions in organic solvents. Depending upon the leaving group employed, the activated polymers can be used to link to amino groups, hydroxyl groups, thiol groups, mercaptans, etc. In preferred aspects, the activated polymers are used to conjugate with amine-containing drugs and biologically activate polypeptides. Examples of such biologically active compounds include but are not limited to anti-infectives such as vancomycin:

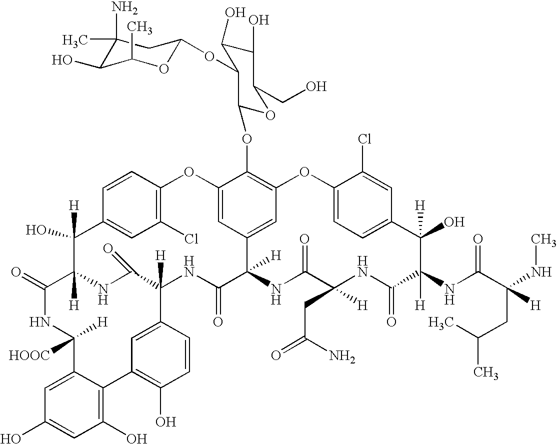

Ara-C (cytosine arabinoside):

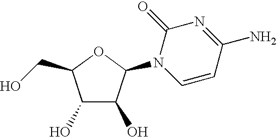

or known derivatives thereof, and gemcitabine:

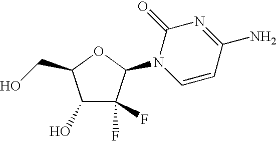

Still other compounds which can be conjugated to the activated polymers of the present invention include as anthracycline compounds including daunorubicin, doxorubicin; p-aminoaniline mustard, melphalan, anti-infectives including, nystatin, etc. The target compounds selected for polymer attachment need not be substantially water-insoluble, although the polymer-based prodrugs of the present invention are especially well suited for delivering such water-insoluble compounds.

Other useful parent compounds include, for example, certain low molecular weight biologically active proteins, enzymes and polypeptides, including peptide glycans and the like having at least one available group for polymer attachment, e.g. an ε-amino, cysteine, thio, N-terminal amino, include materials which have physiological or pharmacological activities as well as those which are able to catalyze reactions in organic solvents. The only other requirement of the amine-containing materials is that they maintain at least some portion of the activity associated with the unmodified protein, enzyme, peptide, etc. either after attachment to the polymeric transport or, if relevant, after the parent compound has been hydrolyzed and released.

Proteins, polypeptides and peptides of interest include, but are not limited to, hemoglobin, serum proteins such as blood factors including Factors VII, VIII, and IX; immunoglobulins, cytokines such as interleukins, i.e. IL-1 through IL-13, etc.,α-,β- and γ-interferons, colony stimulating factors including granulocyte colony stimulating factors and platelet derived growth factors. Other proteins of general biological or therapeutic interest include insulin, plant proteins such as lectins and ricins, tumor necrosis factors and related proteins, growth factors such as transforming growth factors, such as TGF-α's or TGF-β's and epidermal growth factors, hormones, somatomedins, erythropoietin, pigmentary hormones, hypothalamic releasing factors, antidiuretic hormones, prolactin, chorionic gonadotropin, follicle-stimulating hormone, thyroid-stimulating hormone, tissue plasminogen activator, and the like. Immunoglobulins of interest include IgG, IgE, IgM, IgA, IgD and fragments thereof.

Some proteins such as the interleukins, interferons and colony stimulating factors also exist in non-glycosylated form, usually as a result of using recombinant techniques. The non-glycosylated versions are also among the proteins of the present invention.

Enzymes of interest include carbohydrate-specific enzymes, proteolytic enzymes, oxidoreductases, transferases, hydrolases, lyases, isomerases and ligases. Without being limited to particular enzymes, examples of enzymes of interest include asparaginase, arginase, arginine deaminase, adenosine deaminase, superoxide dismutase, endotoxinases, catalases, chymotrypsin, lipases, uricases, adenosine diphosphatase, tyrosinases and bilirubin oxidase. Carbohydrate-specific enzymes of interest include glucose oxidases, glucodases, galactosidases, glucocerebrosidases, glucouronidases, etc.

Also included herein is any portion of a biological polymer demonstrating in vivo bioactivity. This includes amino acid sequences, nucleic acids (DNA, RNA), peptide nucleic acids (PNA), antibody fragments, single chain binding proteins, see, for example U.S. Pat. No. 4,946,778, disclosure of which is incorporated herein by reference, binding molecules including fusions of antibodies or fragments, polyclonal antibodies, monoclonal antibodies and catalytic antibodies.

The proteins or portions thereof can be prepared or isolated by using techniques known to those of ordinary skill in the art such as tissue culture, extraction from animal sources, or by recombinant DNA methodologies. Transgenic sources of the proteins, polypeptides, amino acid sequences and the like are also contemplated. Such materials are obtained from transgenic animals, i.e., mice, pigs, cows, etc., wherein the proteins are expressed in milk, blood or tissues. Transgenic insects and baculovirus expression systems are also contemplated as sources. Moreover, mutant versions of proteins, such as mutant interferons are also within the scope of the invention.

Other proteins of interest are allergen proteins such as ragweed, Antigen E, honeybee venom, mite allergen, and the like. The foregoing is illustrative of the proteins suitable for the present invention. It is to be understood that those proteins, as defined herein, not specifically mentioned but having an available amino group are also intended and are within the scope of the present invention.

In a preferred aspect of the invention, the amino-containing compound is a biologically active compound that is suitable for medicinal or diagnostic use in the treatment of animals, e.g., mammals, including humans, for conditions for which such treatment is desired. The foregoing list is meant to be illustrative and not limiting for the compounds which can be modified. Those of ordinary skill will realize that other such compounds/compositions can be similarly modified without undue experimentation. It is to be understood that those biologically active materials not specifically mentioned but having suitable attachment groups are also intended and are within the scope of the present invention.

The forgoing is illustrative of the biologically active moieties which are suitable for conjugation with the prodrugs of the present invention. It is to be understood that those biologically active materials not specifically mentioned but having suitable amine functional groups are also intended and are with in the scope of the present invention.

Unless stated otherwise, the substituents mentioned herein are reacted in an inert solvent such as tetrahydrofuran (THF), acetonitrile (CH₃CN), methylene chloride (DCM), chloroform (CHCl₃), dimethyl formamide (DMF) or mixtures thereof. The reaction is preferably conducted in the presence of a base, such as dimethylaminopyridine (DMAP), diisopropylethylamine, pyridine, triethylamine, KOH, potassium t-butoxide and NaOH etc. and at a temperature from 0° C. up to about 22° C. (room temperature).

Examples of such activated PEG linkers resulting from the synthesis techniques described herein include but are not limited to:

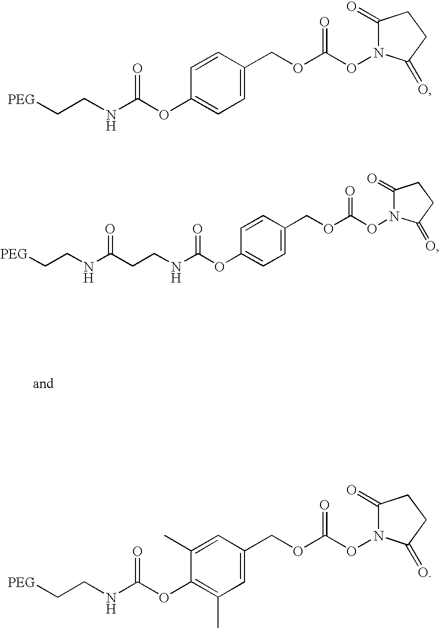

### EXAMPLES

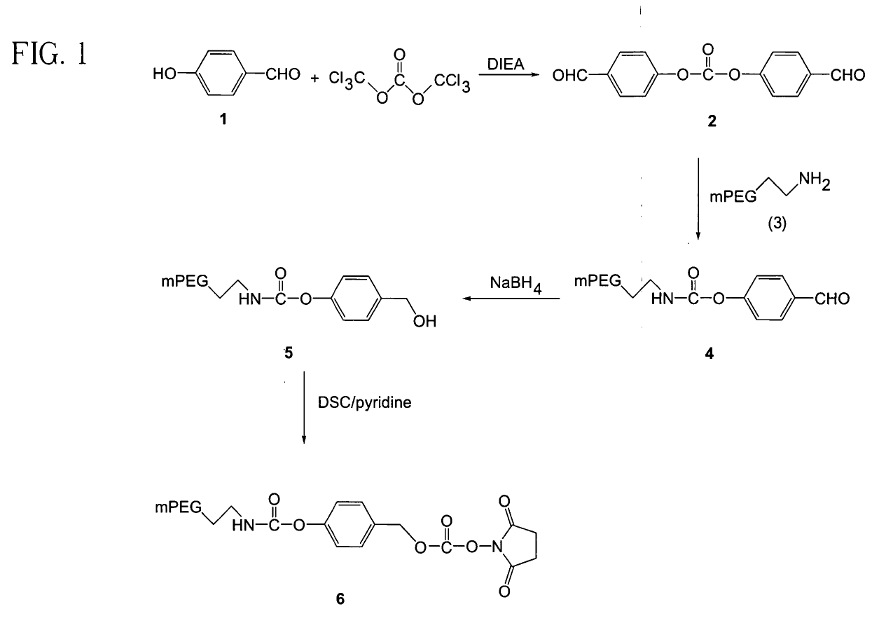

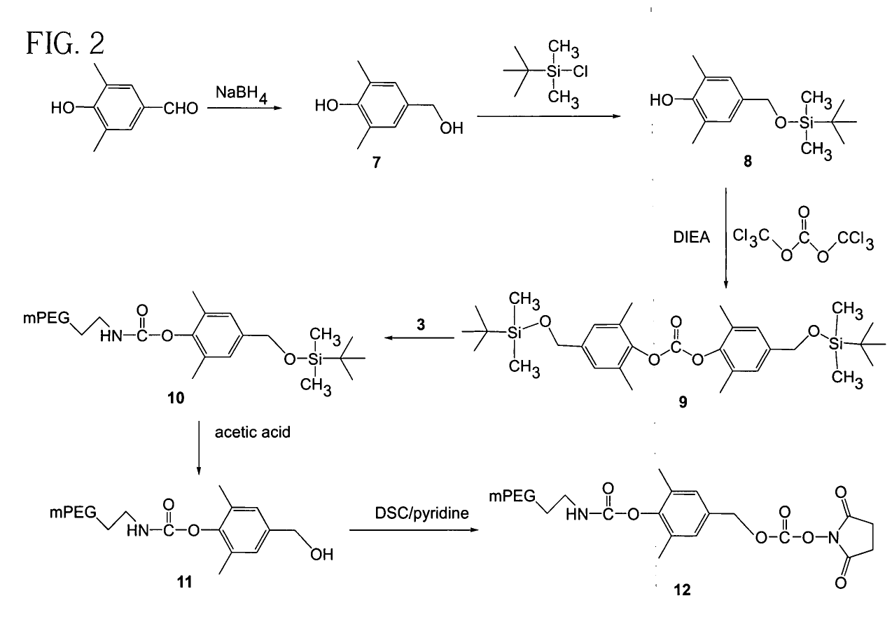

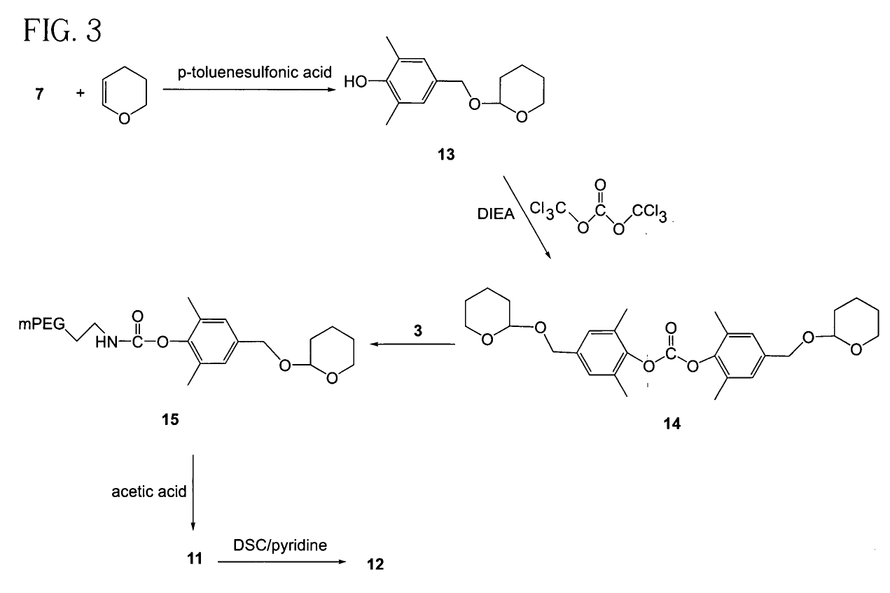

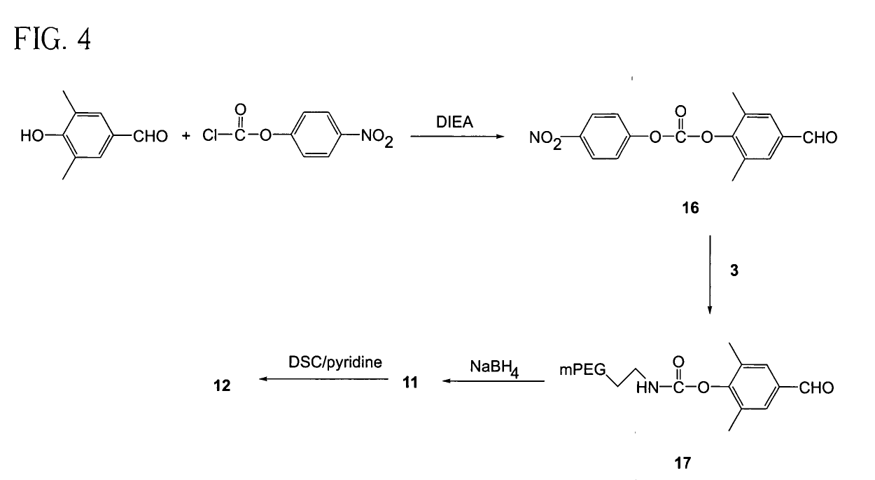

The following examples serve to provide further appreciation of the invention but are not meant in any way to restrict the effective scope of the invention. The underlined and bold face numbers recited in the Examples correspond to those shown in FIGS. 1 to 5.

General Procedures

All reactions were run under an atmosphere of dry nitrogen or argon. Commercial reagents were used without further purification. All PEG compounds were dried under vacuum or by azeotropic distillation from toluene prior to use. NMR spectra were obtained using a Varian Mercury™300 NMR spectrometer and deuterated chloroform as the solvent unless otherwise specified. Chemical shifts (δ) are reported in parts per million (ppm) downfield from tetramethylsilane (TMS).

### Example 1

Compound 2. To a solution of of 4-hydroxybenzaldehyde (1, 1.0 g, 8.2 mmol) and triphosgene (0.34 g, 1.14 mmol) in anhydrous DCM (20 mL) cooled to 15° C. was added diisopropylethylamine (DIEA, 1.2 mL, 6.87 mmol) dropwise over a time period of 5 minutes. The cooling bath was then removed, and the reaction mixture stirred at room temperature for one hour. The solution was washed with 0.1 N HCl and the organic layer was dried (anhydrous sodium sulfate), filtered, and the solvent removed from the filtrate under reduced pressure to give 2 (0.8 g, 2.99 mmol, 73%). ¹³C NMR (67.8 MHz, CDCl₃) δ 190.49, 154.82, 150.37, 134.36, 131.27, 121.42.

### Example 2

Compound 4. A solution of 12 kDa methoxypolyethylene glycol (mPEG) amine (3, 1.1 g, 0.22 mmol), 2 (0.36 g, 1.32 mmol), and DIEA (0.23 mL, 1.32 mmol) in methylene chloride (DCM, 15 mL) was stirred at room temperature for 12 hrs. The solvent was partially removed under reduced pressure, followed by precipitation of the product with ethyl ether. The solid was collected by filtration, washed with ether and crystallized from 2-propanol (IPA, 22 mL) to yield 4 (0.88 g, 80%) of. ¹³C NMR (67.8 MHz, CDCl₃) δ 190.35, 155.52, 153.17, 132.85, 130.62, 121.57, 71.57-69.29 (PEG), 58.65, 40.80.

### Example 3

Compound 5. To a solution of 4 (16.6 g, 3.23 mmol) in methanol (160 mL) cooled to 15° C. was added sodium borohydride (0.2 g, 5.35 mmol) and the resulting mixture allowed to warm to room temperature over a period of 2 hrs, followed by adjusting the pH to 6.5 with 1N HCl. The methanol was removed under reduced pressure, and the residue taken up in water. The pH was lowered to 2.0 with 1.0 N HCl, and the product was extracted from the water with DCM, dried (anhydrous sodium sulfate), filtered, and the solvent partially removed under reduced pressure. The product was precipitated with ethyl ether, collected by filtration, and washed with ethyl ether to yield 5 (14.4 g, 87%). ¹³C NMR (67.8 MHz, CDCl₃) δ 154.39, 149.89, 138.05, 127.36, 121.11, 71.52-69.42 (PEG), 63.81, 58.62, 40.60.

### Example 4

Compound 6. To a solution of 5 (4.0 g, 0.78 mmol), disuccinimidyl-carbonate (DSC, 1.6 g, 6.22 mmol) in DCM (30 mL) cooled to 0° C. was added pyridine (0.25 g, 3.1 mmol) and the resulting mixture stirred at 0° C. for 12 hrs. The solvent was partially removed in vacuo and the product precipitated by addition of ethyl ether, filtered, and crystallized from DCM/ethyl ether to give 6 (2.0 g, 49%). ¹³C NMR (67.8 MHz, CDCl₃) δ 168.39, 154.15, 151.62, 151.34, 129.86, 129.61, 121.76, 71.97-69.54 (PEG), 58.78, 40.84, 25.22.

### Example 5

Compound 7. To a solution of 3,5-dimethyl-4-hydroxybenzaldehyde (5.0 g, 0.33 mol) in methanol (75 mL) cooled to 15° C. was added sodium borohydride (3.8 g, 0.10 mol). The cooling bath was then removed, and the reaction mixture stirred at room temperature for one hour, followed by acidification with 0.1N HCl solution. The solvent was removed under reduced pressure and the residue taken up in water (50 mL), and extracted with DCM. The organic layer was dried (anhydrous sodium sulfate), filtered, and the solvent removed under reduced pressure to yield 7 (2.6 g, 52%). ¹³C NMR (67.8 MHz, CDCl₃) δ 151.83, 132.51, 127.79, 123.24, 65.20, 15.84.

### Example 6

Compound 8. To a solution of 7 (2.0 g, 13.2 mmol) and dimethyl-t-butylsilylchloride (2.2 g, 14.7 mmol) in DCM (20 mL) cooled to 0° C. was added a solution of triethylamine (10 mL, 0.1 mol) in DCM (10 mL) in portions over a period of 1 hr and the reaction mixture stirred at room temperature for an additional 3 hrs. The solvents were evaporated under reduced pressure and the residue dissolved in DCM, which was washed four times with water. The organic layer was dried (anhydrous sodium sulfate), filtered, and the solvent removed from the filtrate in vacuo to give 8 (3.5 g, 100%). ¹³C NMR (67.8 MHz, CDCl₃) δ 151.56, 133.13,127.12, 123.24, 26.13, 18.58, 16.08.

### Example 7

Compound 9. To a solution of 8 (1.0 g, 3.76 mmol) and triphosgene (0.19 g, 0.63 mmol) in anhydrous DCM (15 mL) cooled to 15° C. was added DIEA (0.66 mL, 3.76 mmol) dropwise over a period of 5 minutes. The cooling bath was removed, and the reaction mixture was stirred at room temperature for one hour, followed by washing with 0.1N HCl solution. The organic layer was dried (sodium sulfate), filtered, and the solvent removed from the filtrate under reduced pressure to yield 9 (0.8 g, 73%). ¹³C NMR (67.8 MHz, CDCl₃) δ 148.74, 148.31, 140.14, 129.41, 64.23, 26.00, 18.47, 16.14.

### Example 8

Compound 10. A solution of 3 (1.1 g, 0.22 mmol), 9 (0.37 g, 0.66 mmol), and DIEA (0.12 ml, 0.66 mmol) in DCM (15 mL) was refluxed for 12 hrs. The solvent was partially removed from the reaction mixture in vacuo, followed by precipitation of the product with ether. The solid was collected by filtration, washed with ether and crystallized from IPA (22 mL) to yield 10 (0.85 g, 77%). ¹³C NMR (67.8 MHz, CDCl₃) δ 153.73, 146.53, 137.83, 130.10, 125.71, 64.22, 58.65, 40.83, 25.73, 18.16, 16.07.

### Example 9

Compound 11 (method 1). A solution of 10 (0.8 g, 0.15 mmol) in a mixture of glacial acetic acid (7.5 mL) and water (2.5 mL) is stirred at room temperature for 2 hrs, followed by neutralization with sodium bicarbonate, and extraction with DCM. The organic layer is dried (anhydrous sodium sulfate), filtered, and the solvent removed from the filtrate under reduced pressure to yield 11 (0.7 g, 90%). The structure of 11 is confirmed by ¹³C NMR.

### Example 10

Compound 12. To a solution of 11 (4.0 g, 0.78 mmol), disuccinimidyl-carbonate (DSC, 1.6 g, 6.22 mmol) in DCM (30 mL) cooled to 0° C. is added pyridine (0.25 g, 3.1 mmol) and the resulting mixture stirred at 0° C. for 12 hrs. The solvent is partially removed in vacuo and the product precipitated by addition of ethyl ether, filtered, and crystallized from DCM/ethyl ether to give 12 (2.0 g, 49%). The structure of 12 is confirmed by ¹³C NMR.

### Example 11

Compound 13. A solution of 7 (4.0 g, 0.026 mol), 3,4-dihydro-2H-pyran (2.2 g, 0.026 mol), and p-toluenesulfonic acid (0.1 g, catalyst) in toluene (50 mL) is azeotroped for 2 hrs. The solvent is removed under reduced pressure and the residue purified by column chromatography to give 13. The structure of 13 is confirmed by ¹³C NMR.

### Example 12

Compound 14. To a solution of 13 (1.0 g, 4.2 mmol) and triphosgene (0.15 g, 0.51 mmol) in anhydrous DCM (20 mL) cooled to 15° C. was added DIEA (0.45 mL, 3.52 mmol) dropwise over a period of 5 minutes. The cooling bath is removed, and this reaction mixture is stirred at room temperature for an additional hour, followed by washing with 0.1N HCl solution. The organic layer is dried (anhydrous sodium sulfate), filtered, and the solvent removed from the filtrate under reduced pressure to yield 14 (0.8 g, 80%). The structure of 13 is confirmed by ¹³C NMR.

### Example 13

Compound 15. A solution of 3 (1.1 g, 0.22 mmol), 14 (0.33 g, 0.66 mmol), and DIEA (0.12 mL, 0.66 mmol) in DCM (15 mL) is stirred at room temperature for 12 hrs. The solvent is partially removed under reduced pressure, followed by precipitation of the product with ether. The solid is collected by filtration, washed with ether and crystallized from IPA to yield 15 (0.9 g, 82%). The structure of 13 is confirmed by ¹³C NMR.

### Example 14

Compound 11 (method 2). A solution of 15 (0.8 g, 0.15 mmol) in a mixture of glacial acetic acid (7.5 mL) and water (2.5 mL) is stirred at room temperature for 2 hrs, followed by neutralization with sodium bicarbonate, and extraction with DCM. The organic layer is dried (anhydrous sodium sulfate), filtered, and the solvent removed from the filtrate under reduced pressure to yield 11 (0.7 g, 90%). The structure of 11 is confirmed by ¹³C NMR. Formulation of 12 proceeds according to Example 10.

### Example 15

Compound 16. To a solution of 3,5-dimethyl-4-hydroxybenzaldehyde (1.0 g, 6.7 mmol) and 4-nitrophenylchloroformate (1.35 g, 6.7 mmol) in anhydrous DCM (20 mL) cooled to 15° C. was added DIEA (1.16 mL, 6.7 mmol) dropwise over a period of 5 minutes. The cooling bath was removed, and the reaction mixture stirred at room temperature for one hour, followed by washing with 0.1N HCl solution. The organic layer was dried over anhydrous sodium sulfate, filtered, and the solvent removed from the filtrate in vacuo to give 16 (1.9 g, 90%). ¹³C NMR (67.8 MHz, CDCl₃) δ 191.03, 155.00, 152.14, 149.58, 145.60, 134.53, 131.27, 130.40, 125.41, 121.47, 16.23.

### Example 16

Compound 17. A solution of 3 (1.1 g, 0.22 mmol), 16 (0.49 g, 1.54 mmoe), and DIEA (0.27 mL, 1.54 mmol) in DCM (15 mL) was stirred at room temperature for 12 hrs. The solvent was partially removed in vacuo, followed by precipitation of the product with ether. The solid was collected by filtration, washed with ether and crystallized from 2IPA to yield product 17 (0.85 g, 77%). ¹³C NMR (67.8 MHz, CDCl₃) δ 190.88, 152.72, 152.60, 133.14, 131.76, 129.52, 71.46-69.53(PEG), 58.59, 40.80, 16.22.

### Example 17

Compound 18. A solution of 3,5-dimethyl-4-hydroxybenzaldehyde (4.0 g, 26.7 mmol) and triphosgene (1.32 g, 4.44 mmol) in anhydrous DCM (20 mL) cooled to 15° C. was added DIEA (4.6 mL, 26.7 mmol) dropwise over a period of 5 minutes. The cooling bath was removed, and the reaction mixture stirred at room temperature for one hour, followed by washing with 0.1N HCl solution. The organic layer was dried over anhydrous sodium sulfate, filtered, and the solvent removed under reduced pressure and the residue crystallized from IPA to give 18 (1.2 g, 28%). ¹³C NMR (67.8 MHz, CDCl₃) δ 191.06, 152.26, 149.38, 134.46, 131.39, 130.41, 16.22.

### Example 18

Compound 19. A solution of 3 (1.1 g, 0.22 mmol), 18 (0.20 g, 0.6 mmol), and DIEA (0.11 mL, 0.6 mmol) in DCM (15 mL) was stirred at room temperature for 12 hrs. The solvent was partially removed under reduced pressure, followed by precipitation of the product with ether. The solid was collected by filtration, washed with ether and crystallized from IPA to 19 (0.6 g, 57%). ¹³C NMR (67.8 MHz, CDCl₃) δ 190.56,161.01, 151.91, 149.59, 149.24,149.11, 133.92, 133.75, 130.97, 129.89, 128.16.

### Example 19

Compound 20. To a solution of 18 (0.11 g, 0.34 mmol) in methanol (11 mL) cooled to 15° C. was added sodium borohydride (0.028 g, 0.74 mmol). The cooling bath was removed, and the reaction mixture stirred at room temperature for one hour, followed by acidification with 0.1N HCl solution. The solvent removed from the filtrate in vacuo, the residue taken up in water (20 mL), and extracted with DCM. The organic layer was dried over anhydrous sodium sulfate, filtered, and the solvent removed under reduced pressure to give 20 (0.09 g, 81%). ¹³C NMR (67.8 MHz, CDCl₃) δ 150.96, 146.99, 139.03, 129.71, 127.12, 63.73, 15.78.

### Example 20

Compound 21. A solution of 20 (0.13 g, 0.40 mmol), n-hexylamine (1.0 g, 9.9 mmol) and DMAP (0.04 g, 0.33 mmol) in DMF (5 mL0 was stirred at 70° C. for 18 hrs. The solvent was removed in vacuo and the residue taken up in DCM and washed three times with 0.1 N HCl. The organic layer was dried over anhydrous sodium sulfate, filtered, and the solvent removed under reduced pressure to yield crude product, which was purified by column chromatography on silica gel to yield pure 21 (0.055 g, 50%). ¹³C NMR (67.8 MHz, CDCl₃) δ 158.42, 151.91, 129.03, 128.33, 127.63, 65.10, 40.63, 31.58, 30.24, 26.62, 22.62, 16.09, 14.06.

### Example 21

Compound 11 (method 3). A solution of 20 (0.13 g, 0.40 mmol), mPEG₅₀₀₀ (5.0 g, 10.0 mmol) and DMAP (0.04 g, 0.33 mmol) in DMF (5 mL0 was stirred at 70° C. for 18 hrs. The solvent was removed in vacuo and the residue taken up in DCM and washed three times with 0.1 N HCl. The organic layer was dried over anhydrous sodium sulfate, filtered, and the solvent removed under reduced pressure to yield crude product, which was purified by column chromatography on silica gel to yield pure 11 (0.055 g, 50%). ¹³C NMR (67.8 MHz, CDCl₃) δ 158.42, 151.91, 129.03, 128.33, 127.63, 65.10, 40.63, 31.58, 30.24, 26.62, 22.62, 16.09, 14.06. The structure of 11 is confirmed by ¹³C NMR. Formulation of 12 proceeds according to Example 10.

### Example 22

Compound 22. A solution of 6 (2.0 g, 0.378 mmol), AraC (0.191 g, 0.756 mmol), and DMAP (0.093 g, 0.756 mmol) in anhydrous DMF/DCM (20 mL/220 mL) is stirred at room temperature for 12 hrs. The solvents are partially removed under reduced pressure and the final product is precipitated with ethyl ether (80 mL). The solid is filtered and recrystallized from DMF/methanol (35 mL/25 mL) to give 22, shown below. The structure of 22 is confirmed by ¹³C NMR.

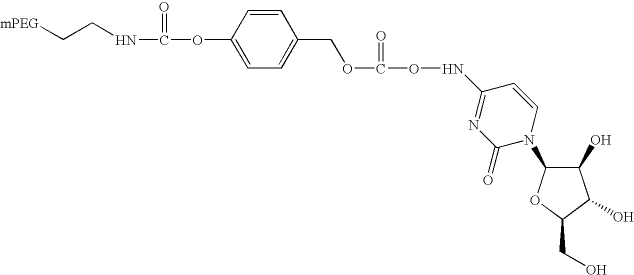

### Example 23

Compound 23. A solution of 12 (2.0 g, 0.376 mmol), doxorubicin hydrochloride (0.436 g, 0.751 mmol), and DMAP (0.092 g, 0.751 mmol) in anhydrous DMF/DCM (20 mL/20 mL) is stirred at room temperature for 12 hrs. The solvents are partially removed under reduced pressure and the final product is precipitated with ethyl ether (80 mL). The solid is filtered and recrystallized from DMF/methanol (35 mL/25 mL) to give 23, shown below. The structure of 23 is confirmed by ¹³C NMR.

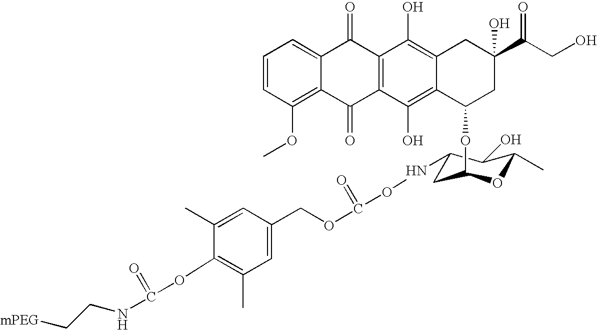

Other embodiments of the invention will be apparent to one skilled in the art from a consideration of this specification or practice of the invention disclosed herein. It is intended that the specification and examples be considered as exemplary only, with the true scope and spirit of the invention being indicated by the following claims.

What is claimed is:

1. A compound having the formula: 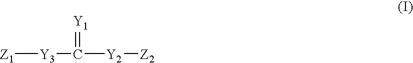
wherein:
Y₁₋₃ are independently O, S or NR₁;
Z₁ and Z₂ are independently selected substituted or unsubstituted aromatic hydrocarbons or substituted or unsubstituted heterocyclic aromatic groups containing an aldehyde or protecting group, and
R₁ is selected from the group consisting of hydrogen, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxys, phenoxys and C₁₋₆ heteroalkoxys.

2. The compound of claim 1, wherein
Z₁ is 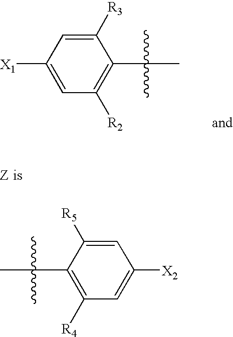
wherein:
X₁ and X₂ are independently selected from the group consisting of
—CHO,
—NO₂, 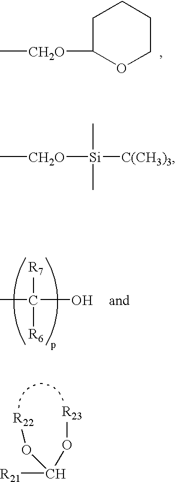
wherein
R₂₋₇ and R₂₁ are independently selected from the group consisting of hydrogen, C₁₋₆ alkyls, C₃₋₈ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxys, phenoxys and C₁₋₆ heteroalkoxys;
R₂₂ and R₂₃ are selected from the same group which defines R₂ and optionally together form a heterocyclic group; and
p is a positive integer.

3. The compound of claim 2 wherein, Y₁₋₃ are each O, R₃₋₆ are independently one of hydrogen or a C₁₋₆ alkyl, and Z₂ is the same as Z₁.

4. The compound of claim 1 having the formula:

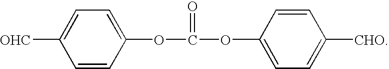

5. The compound of claim 1 having the formula:

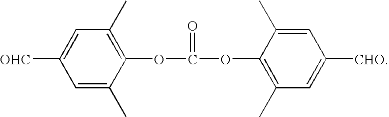

6. The compound of claim 1 having the formula:

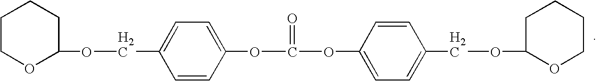

7. The compound of claim 1 having the formula:

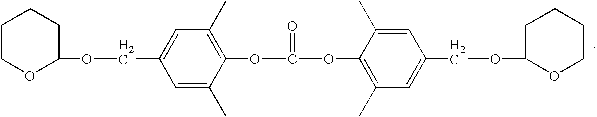

8. The compound of claim 1 having the formula:

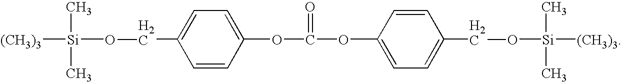

9. The compound of claim 1 having the formula:

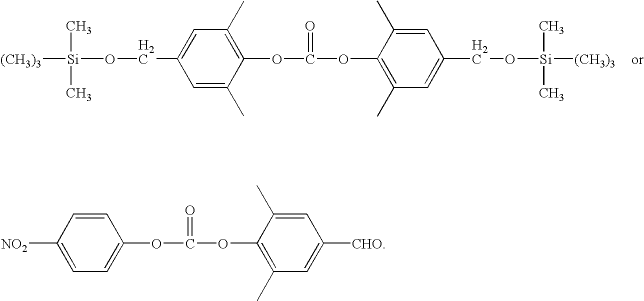

10. A method of preparing an activated nucleophile, comprising:
a) reacting a compound having the formula: 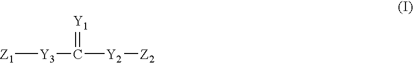
wherein:
Y₁₋₃ are independently O, S or NR₁;
Z₁ and Z₂ are independently selected substituted or unsubstituted aromatic hydrocarbons or substituted or unsubstituted heterocyclic aromatic groups containing an aldehyde or protecting group; and
R₁ is selected from the group consisting of hydrogen, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxys, phenoxys and C₁₋₆ heteroalkoxys;
with a strong nucleophile under conditions sufficient to form a compound of formula (II): 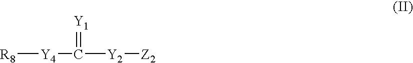
wherein:
R₈ is a residue of a strong nucleophile;
Y₄ is NR₂₀, O or S;
Y₁₋₂ are independently O, S or NR₁;
Z₂ is a substituted or unsubstituted aromatic hydrocarbon or substituted or unsubstituted heterocyclic aromatic group containing an aldehyde or protecting group; and
R₁ and R₂₀ are independently selected from the group consisting of hydrogen, C₁₋₆ alkyls, C₃₋₈ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxys, phenoxys and C₁₋₆ heteroalkoxys.

11. The method of claim 10, wherein Z₂ is: 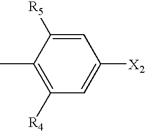
wherein:
R₄₋₅ are independently selected from the group consisting of hydrogen, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxys, phenoxys and C₁₋₆ heteroalkoxys; and
X₂ is an aldehyde or protecting group.

12. The method of claim 11, wherein X₂ is CHO.

13. The method of claim 11, wherein X₂ is

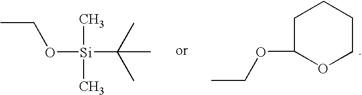

14. The method of claim 10, further comprising converting X₂ to an alcohol and thereby forming a compound of the formula: 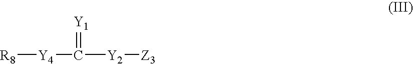
wherein Z₃ is substituted or unsubstituted aromatic hydrocarbon or substituted or unsubstituted heterocyclic aromatic group substituted with 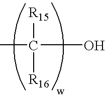
wherein
R₁₅₋₁₆ are independently selected from the group consisting of hydrogen, C₁₋₆ alkyls, C₃₋₈ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxys, phenoxys and C₁₋₆ heteroalkoxys; and
w is a positive integer.

15. The method of claim 14, wherein p is 1.

16. The method of claim 14, further comprising reacting said compound of formula (III) with a moiety containing a leaving group under conditions sufficient to form an activated polymer of the formula: 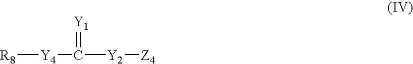
wherein
R₈ is a residue of a strong nucleophile;
Y₄ is NR₂₀, O or S;
Y₁₋₂ are independently O, S or NR₁;
R₁ and R₂₀ are independently selected from the group consisting of hydrogen, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxys, phenoxys and C₁₋₆ heteroalkoxys; and
Z₄ is a leaving group.

17. The method of claim 16, wherein said moiety containing a leaving group is selected from the group consisting of disuccinimidyl carbonate and N-hydroxypthalamide.

18. The method of claim 10, wherein R₈ comprises a polyalkylene oxide residue.

19. The method of claim 18, wherein R₈ is a polyethylene glycol residue.

20. The method of claim 18, wherein R₈ comprises —O—(CH₂CH₂O)ₓ and x is the degree of polymerization.

21. The method of claim 18, wherein R₈ has a weight average molecular weight of from about 20,000 to about 100,000.

22. The method of claim 10, wherein R₈ has a weight average molecular weight of from about 25,000 to about 60,000.

23. The method of claim 16, further comprising reacting the activated polymer of formula IV with a biologically active compound to form a polymer conjugate.

24. A method of preparing an activated nucleophile, comprising:
a) reacting a compound having the formula: 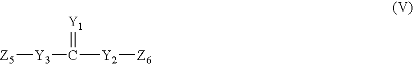
wherein:
Y₁₋₃ are independently O, S or NR₁;
Z₅ and Z₆ are independently selected substituted or unsubstituted aromatic hydrocarbons or substituted or unsubstituted heterocyclic aromatic groups, substituted with 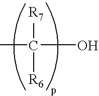
wherein
R₁ and R₆₋₇ are independently selected from the group consisting of hydrogen, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxys, phenoxys and C₁₋₆ heteroalkoxys;
p is a positive integer; and
with a nucleophile under conditions sufficient to form a compound of Formula (IIa): 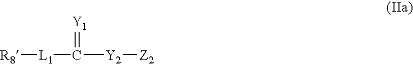
wherein:
R₈′ is a nucleophile residue;
L₁ is a bifunctional linker
Y₁₋₂ are independently O, S or NR₁;
Z₆ is a substituted or unsubstituted aromatic hydrocarbon or substituted or unsubstituted heterocyclic aromatic group, substituted with 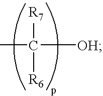
and
R₁, R₆₋₇, and R₂₀ are independently selected from the group consisting of hydrogen, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxys, phenoxys and C₁₋₆ heteroalkoxys.
